(19) United States
(12) Patent Application Publication
(10) Pub. No.: US 2026/0212969 A1
(43) Pub. Date: Jul. 23, 2026

(54) COMPUTER-IMPLEMENTED SYSTEM AND METHOD FOR PROVIDING OPTIMIZED PROCESS IN CLINICAL RESEARCH

(71) Applicant: HILM LLC, Burr Ridge, IL (US)

(72) Inventor: Nusrat Ahmed, Burr Ridge, IL (US)

(73) Assignee: HILM LLC, Burr Ridge, IL (US)

(21) Appl. No.: 19/563,697

(22) Filed: Mar. 11, 2026

Related U.S. Application Data
(63) Continuation-in-part of application No. 17/833,922, filed on Jun. 7, 2022, now abandoned.

(51) Int. Cl. G16H 10/20; G06Q 10/0631; G16H 10/60; G16H 40/20

(52) U.S. Cl. CPC G16H 10/20; G06Q 10/06312; G16H 10/60; G16H 40/20

(57) ABSTRACT

A system and method for autonomously optimizing clinical research processes through a distributed multi-agent artificial intelligence architecture. One or more processors comprising a neural processing hub and an AI inference accelerator execute a research process optimizing module across a distributed network of computing devices. An automated enrolment and retention module parses unstructured medical records using Natural Language Processing and generates high-dimensional semantic vector embeddings stored in a persistent vector database. A list generating module executes a Transformer-based Large Language Model to perform semantic vector matching against clinical study protocol criteria to generate a ranked list of qualified research participants. An integrated Generative AI chatbot facilitates participant engagement and informed consent. A Reinforcement Learning agent autonomously assigns principal investigators for screening. A primary source data capturing module utilizes Convolutional Neural Networks for automated Optical Character Recognition and deep learning anomaly detection. A financial module performs autonomous reconciliation and invoice generation with verified data recorded on a distributed ledger.

### CROSS-REFERENCE TO RELATED APPLICATIONS

The present application claims priority to U.S. Provisional Application No. 17/833,922 filed Jun. 7, 2022, titled “COMPUTER-IMPLEMENTED SYSTEM AND METHOD FOR PROVIDING OPTIMIZED PROCESS IN CLINICAL RESEARCH,” which is hereby incorporated by reference in its entirety.

### TECHNICAL FIELD

The embodiments generally relate to artificial intelligence systems and methods for clinical research informatics, and more particularly to a multi-agent orchestration framework employing large language models, reinforcement learning, and computer vision to autonomously manage participant recruitment, regulatory compliance, and financial reconciliation across distributed clinical trial environments.

### BACKGROUND

Clinical research determines the safety and efficacy of new drugs, treatments, medical devices, and diagnostic products before they reach the general population. The clinical trial lifecycle involves multiple coordinated stages, including participant identification and recruitment, regulatory document management, screening and data capture by principal investigators, and financial reconciliation between research sites and sponsors. Each stage requires the exchange of detailed medical, regulatory, and financial information among research participants, site managers, principal investigators, and sponsoring organizations, often across geographically distributed locations.

Conventional clinical trial management systems rely on Electronic Data Capture (EDC) platforms and related software tools to digitize portions of this workflow. These systems typically require site staff to manually review patient charts and medical records against a study protocol's inclusion and exclusion criteria to identify potential research participants. Because clinical trial eligibility criteria are often complex and expressed in nuanced medical language, site coordinators frequently perform keyword-based searches across electronic health records or manually sift through paper charts to locate candidates. This process is time-intensive, dependent on individual reviewer expertise, and limited in its ability to account for the broader clinical context captured in unstructured clinician narratives such as progress notes, consultation reports, and procedure notes.

Once participants are identified and enrolled, conventional systems manage screening appointments, regulatory documentation, and source data verification through a combination of manual scheduling workflows, physical document binders, and fragmented software tools. Site staff typically coordinate investigator assignments, appointment times, and participant reminders through email, phone calls, or standalone scheduling applications that operate independently from the trial management platform. Regulatory documents, including financial disclosure forms, delegation logs, and investigator credentials, are commonly maintained in paper-based filing systems that require manual organization, physical storage, and periodic scanning for remote sponsor review. During screening visits, principal investigators capture clinical observations and verify laboratory reports and medical imaging through manual review and handwritten or typed entries into electronic case report forms.

Financial reconciliation between research sites and trial sponsors presents additional operational challenges. Site managers typically track completed study visits and billable procedures using spreadsheets or standalone accounting tools that are not integrated with the clinical data capture systems. Reconciling performed procedures against contractual payment schedules requires manual cross-referencing, which introduces the potential for billing discrepancies, delayed invoicing, and incomplete capture of reimbursable line items. Sponsors, in turn, rely on periodic site reports and manual audits to verify financial claims and assess overall trial progress, which can limit real-time visibility into enrollment velocity and site performance.

These conventional approaches, while functional, involve substantial manual coordination across disconnected systems and stakeholders. The reliance on human-driven processes for participant matching, document management, data verification, and financial tracking creates operational bottlenecks that can slow trial timelines and increase administrative overhead for clinical research organizations.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

The present disclosure provides a system and computer-implemented method for autonomously optimizing clinical research processes through a distributed multi-agent artificial intelligence architecture. The system comprises one or more processors including a neural processing hub and an AI inference accelerator coupled to a non-transitory memory storing a research process optimizing module. The research process optimizing module operates across a distributed network of computing devices used by research participants, site managers, principal investigators, and sponsors. An automated enrolment and retention module receives unstructured health history and medical records and parses them using a Natural Language Processing model to transform the records into high-dimensional semantic vector embeddings stored in a persistent vector database. A list generating module executes a Transformer-based Large Language Model to perform semantic vector matching of the stored embeddings against inclusion criteria and exclusion criteria of a clinical research study protocol, thereby generating a ranked list of qualified research participants based on the clinical context of the medical records rather than keyword-based searches alone.

The system further comprises an integrated Generative AI chatbot configured to interact with qualified research participants to provide protocol-specific clinical decision support, facilitate electronic signing of informed consent documents, and assist with scheduling screening appointments. An appointment scheduling module employs a Reinforcement Learning agent to autonomously assign principal investigators for screening based on investigator therapeutic expertise, investigator availability, and real-time site resource states. A text reminder generating module driven by Reinforcement Learning agents utilizes Predictive Sentiment Analysis to determine the optimal timing and semantic tone of participant notifications to support enrollment velocity and long-term retention. A distributed ledger e-regulatory module utilizes Autonomous AI-Categorization Agents to ingest, digitize, classify, and store regulatory records including financial disclosure forms, delegation logs, and curriculum vitae within an electronic storage system, replacing manual paper-based document management at the research site.

During screening visits, a primary source data capturing module captures screening data in real-time on the principal investigator computing device. The primary source data capturing module executes Convolutional Neural Networks and Computer Vision for automated Optical Character Recognition to classify and verify laboratory reports and medical imaging, and employs deep learning anomaly detection to flag protocol deviations. Verified screening data and investigator digital sign-offs are recorded on a distributed ledger to ensure adherence to ALCOA principles. The primary source data capturing module provides the principal investigator with real-time protocol compliance guidance during history and physical examinations, reducing the potential for manual data entry errors and protocol deviations that can arise in conventional electronic data capture workflows.

A financial module orchestrated by autonomous AI agents collects verified screening data from the distributed ledger and autonomously cross-references completed research participant visits against digital sponsor contracts using Reinforcement Learning to calculate accounts receivable and eliminate manual billing discrepancies. A data review and reports generating module generates screening reports representing the financial value owed to the research site and provides a Predictive Analytics dashboard for both the site manager and the sponsor to review screening data, trial velocity metrics, and enrollment quality in real-time. An invoice generating module autonomously generates digital invoices based on the screening reports and transmits the invoices to the sponsor computing device, with each transaction recorded on the distributed ledger to ensure immutable auditability. This integrated financial orchestration addresses the operational bottlenecks associated with manual spreadsheet-based reconciliation and periodic reporting by providing continuous, real-time visibility into site financial health and overall trial performance across the distributed network.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a system architecture diagram of a distributed multi-agent AI framework for clinical research optimization, according to some embodiments.

FIG. 2 illustrates an alternative system architecture diagram with the persistent vector database and distributed ledger as external data stores, according to some embodiments.

FIG. 3 illustrates a detailed view of the data ingestion, semantic transformation, and participant matching pipeline, according to some embodiments.

FIG. 4 illustrates a clinical research optimization system depicting bidirectional data flow between computing devices and the research process optimizing platform, according to some embodiments.

FIG. 5 illustrates a method for autonomous financial reconciliation and digital invoice generation from verified screening data, according to some embodiments.

FIG. 6 illustrates a serial clinical data processing pipeline from unstructured medical record ingestion through real-time screening data capture, according to some embodiments.

FIG. 7 illustrates an end-to-end method for providing an autonomously optimized multi-agent process in clinical research, according to some embodiments.

FIG. 8 illustrates a research process optimizing platform with a hybrid cloud and local deployment architecture, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

A system for autonomously optimizing clinical research processes comprises one or more processors coupled to a non-transitory memory and communicatively connected to a distributed network of computing devices. The one or more processors may include a neural processing hub configured for parallel execution of neural network inference operations and an AI inference accelerator configured for high-dimensional tensor operations such as matrix multiplications and convolution computations used in deep learning workloads. The neural processing hub may be implemented as a multi-core central processing unit with instruction sets optimized for floating-point arithmetic and parallel thread execution, or as a dedicated neural processing unit with fixed-function hardware for multiply-accumulate operations. The AI inference accelerator may be implemented as a graphics processing unit, a field-programmable gate array, or an application-specific integrated circuit designed to accelerate the forward-pass computations of trained neural network models. The one or more processors execute instructions stored in the non-transitory memory to carry out the autonomous operations described herein.

The non-transitory memory stores a research process optimizing module that orchestrates the clinical research workflow. The non-transitory memory may comprise volatile memory such as random access memory that provides a low-latency runtime environment for executing neural network models and reinforcement learning agents, and non-volatile memory such as solid-state drives or hard disk drives that provide persistent storage for encrypted medical data, trained model weights, and regulatory records. The non-transitory memory further hosts a persistent vector database and a distributed ledger. The persistent vector database stores and indexes research participants' information as high-dimensional semantic vector embeddings, which are fixed-length numerical representations of variable-length text that capture semantic relationships between medical concepts. The persistent vector database may employ approximate nearest neighbor search algorithms, such as hierarchical navigable small world graphs or inverted file indexes with product quantization, to retrieve semantically similar records with sub-linear query complexity. The distributed ledger maintains an append-only, cryptographically linked chain of records that stores verified clinical data captures, investigator sign-offs, regulatory documents, and financial transactions. Each record in the distributed ledger includes a cryptographic hash of the preceding record, a timestamp, and the recorded data, such that any modification to a prior record would invalidate the hash chain and be detectable during audit. This structure provides immutable auditability and supports adherence to ALCOA principles, which require that clinical data be Attributable, Legible, Contemporaneous, Original, and Accurate.

The distributed network of computing devices includes a research participant computing device, a research site manager computing device, a principal investigator computing device, and a sponsor computing device. Each computing device may be a smartphone, tablet, laptop, or desktop computer equipped with a display, an input interface, a network interface, and one or more local processors and memory. The computing devices communicate over a network that may include wired connections such as Ethernet, wireless connections such as Wi-Fi or cellular networks including 5G, or combinations thereof. The research process optimizing module may be deployed as a cloud-hosted application accessible through a web browser or native mobile application on each computing device, or portions of the module may execute locally on each device with synchronization managed through the network. A data ingest and registration module authenticates users across the distributed network. The data ingest and registration module may employ biometric AI and identity verification agents that authenticate users through fingerprint scanning, facial recognition, or iris scanning using trained convolutional neural network classifiers that compare captured biometric samples against enrolled templates. Upon successful authentication, the data ingest and registration module stores identity credentials as immutable records within the distributed ledger and assigns role-based access permissions that control which data layers each user type may access within the research process optimizing module.

The research process optimizing module comprises an automated enrolment and retention module that receives one or more research participants' information from the research participant computing device or the research site manager computing device. The research participants' information may comprise unstructured health history, clinical medical reports, longitudinal diagnosis reports, medication lists, progress notes, consultation reports, procedure notes, laboratory reports, and medical imaging data. The automated enrolment and retention module parses this unstructured information using a Natural Language Processing model to autonomously identify participant eligibility. The NLP model may comprise a pipeline of text processing stages including tokenization, named entity recognition, relation extraction, and negation detection. Tokenization segments raw clinical text into subword units using a byte-pair encoding or SentencePiece vocabulary. Named entity recognition identifies and classifies medical entities such as diagnoses, medications, procedures, anatomical sites, and laboratory values within the tokenized text. The named entity recognition component may be implemented as a bidirectional long short-term memory network with a conditional random field output layer, or as a fine-tuned Transformer encoder that produces token-level classification labels. Relation extraction identifies relationships between recognized entities, such as a medication being prescribed for a specific diagnosis or a laboratory value being associated with a particular organ system. Negation detection identifies negated clinical findings, such as distinguishing “no evidence of diabetes” from “diabetes,” to avoid false-positive eligibility determinations. The NLP model applies these processing stages to transform the unstructured research participants' information into structured representations that capture the clinical semantics of the source documents.

The automated enrolment and retention module then generates high-dimensional semantic vector embeddings from the structured representations. The module may employ a Transformer-based encoder model, such as a model based on the Bidirectional Encoder Representations from Transformers architecture or a domain-specific variant pre-trained on biomedical and clinical text corpora, to produce dense vector representations for each research participant's clinical profile. The Transformer encoder processes tokenized input sequences through a stack of self-attention layers, where each self-attention layer computes attention weights over all positions in the input sequence to capture long-range dependencies between clinical concepts. The output of the final encoder layer is pooled, for example by extracting the representation at a designated classification token position or by computing a mean across all token positions, to produce a single fixed-length embedding vector for the input document. The module generates one embedding vector per clinical document or per participant profile and stores these vectors in the persistent vector database. The persistent vector database indexes the embedding vectors using a spatial indexing structure that supports efficient similarity search based on distance metrics such as cosine similarity or Euclidean distance.

A list generating module executes a Transformer-based Large Language Model on the one or more processors to generate a ranked list of qualified research participants. The list generating module receives as input the inclusion criteria and exclusion criteria of a clinical research study protocol, which may be expressed as free-text descriptions authored by a sponsor or protocol designer. The list generating module transforms these protocol criteria into semantic vector embeddings using the same or a compatible Transformer-based encoder that produced the participant embeddings. The list generating module then performs semantic vector matching by computing similarity scores between the protocol criteria embeddings and the participant profile embeddings stored in the persistent vector database. This semantic matching approach identifies participants whose clinical profiles are contextually aligned with the protocol requirements based on the meaning and relationships captured in the embedding space, rather than relying solely on exact keyword matches against structured diagnosis codes or medication names. The list generating module ranks participants by their aggregate similarity scores and outputs a ranked list of qualified research participants. The list generating module may also apply threshold filtering to exclude participants whose similarity scores fall below a configurable minimum, or whose records contain negated findings or contraindicated conditions identified during the NLP processing stage. The persistent vector database may further enable Predictive Analytics models executed by the AI inference accelerator to forecast recruitment velocity based on historical enrollment patterns and to identify research participants at high risk of dropout by analyzing temporal patterns in engagement data, appointment adherence, and sentiment indicators.

An integrated Generative AI chatbot interacts with qualified research participants through the research participant computing device. The chatbot may be implemented as an autoregressive Transformer-based language model that generates natural language responses conditioned on a conversational context window comprising the participant's prior messages, protocol-specific reference material, and system instructions that constrain the chatbot's responses to protocol-relevant information. The chatbot provides protocol-specific clinical decision support by answering participant questions about study procedures, visit schedules, potential risks and benefits, and eligibility requirements. The chatbot assists participants through the process of reading and providing electronic signatures on informed consent documents by presenting consent content in a conversational format and confirming participant understanding before collecting a digital signature. The AI inference accelerator verifies electronic signatures, and verified consent records are stored on the distributed ledger. The chatbot also facilitates the collection of additional health context from participants, such as updated medication lists or recent medical events, which the automated enrolment and retention module processes and stores as updated embedding vectors in the persistent vector database.

An appointment scheduling module manages the scheduling of screening appointments between qualified research participants and principal investigators. The appointment scheduling module employs a Reinforcement Learning agent to autonomously assign one or more principal investigators for screening based on investigator therapeutic expertise, investigator availability, and real-time site resource states. The RL agent may be implemented as a deep Q-network or a policy gradient agent that operates over a state space, an action space, and a reward function. The state space encodes the current configuration of the research site, including the number of pending screening appointments, the availability schedule of each principal investigator, the therapeutic specialization of each investigator, the complexity of each pending participant's clinical profile, and the current utilization of examination rooms and laboratory facilities. The action space comprises the set of possible investigator-to-appointment assignments for each scheduling decision. The reward function provides a positive signal when an assignment results in a completed screening within a target time window and a negative signal when an assignment results in a scheduling conflict, an investigator mismatch with the participant's therapeutic area, or a participant cancellation. The RL agent is trained through interaction with a simulation environment that models site operations based on historical scheduling data, and the trained policy is deployed to make real-time assignment decisions as new appointments are requested. The appointment scheduling module receives scheduling requests from participants through the integrated Generative AI chatbot and transmits confirmed appointments to the research site manager computing device.

A text reminder generating module driven by Reinforcement Learning agents sends notifications to the research participant computing device to support enrollment velocity and participant retention. The text reminder generating module utilizes Predictive Sentiment Analysis to determine the optimal timing and semantic tone of each notification. The Predictive Sentiment Analysis component may be implemented as a classification model, such as a fine-tuned Transformer encoder, trained on labeled datasets of participant communication responses to predict the likelihood that a given message phrasing and delivery time will produce a positive engagement outcome, such as an appointment confirmation or a response to a study interest inquiry. The RL agents optimize a notification policy over a state space that includes the participant's interaction history, the time elapsed since the last communication, the participant's historical response patterns, and the current stage of the enrollment process. The action space includes message timing options and message tone categories, and the reward function provides a positive signal for participant engagement actions such as appointment confirmations or chatbot interactions and a negative signal for non-responses or opt-out requests. Participants may opt-in to receive these AI-managed automated reminders, and the RL agents dynamically adjust the scheduling frequency and reminder cadence based on the participant's ongoing interaction patterns.

A primary source data capturing module captures screening data of research participants on the principal investigator computing device in real-time during screening visits at a research site location. The primary source data capturing module executes Convolutional Neural Networks and Computer Vision for automated Optical Character Recognition to classify and verify laboratory reports, X-rays, and medical imaging. The OCR pipeline may comprise a text detection stage that identifies regions of text within a scanned document or photograph using a CNN-based object detection model, followed by a text recognition stage that converts the detected regions into machine-readable character sequences using a CNN encoder coupled with a recurrent neural network decoder or a Transformer-based sequence model. The primary source data capturing module applies domain-specific post-processing rules to extract structured data fields from the recognized text, such as laboratory analyte names, reference ranges, measured values, and units. For medical imaging such as X-rays, the module may employ a CNN-based image classification model trained on labeled radiological datasets to flag imaging findings relevant to the study protocol. The primary source data capturing module further employs deep learning anomaly detection to flag protocol deviations in real-time. The anomaly detection component may be implemented as an autoencoder neural network trained on compliant screening records, where the reconstruction error for a new screening record serves as an anomaly score, with records exceeding a configurable threshold flagged for investigator review. The primary source data capturing module provides the principal investigator with real-time protocol compliance guidance by displaying flagged anomalies and validation alerts on the principal investigator computing device as screening data is entered or ingested. Verified screening data and investigator digital sign-offs are recorded on the distributed ledger to maintain an immutable audit trail consistent with ALCOA principles.

A distributed ledger e-regulatory module manages regulatory documents associated with the clinical research study. The e-regulatory module utilizes Autonomous AI-Categorization Agents to ingest, digitize, classify, and store regulatory records including financial disclosure forms, delegation logs, curriculum vitae, and institutional review board communications. The Autonomous AI-Categorization Agents may be implemented as a document classification pipeline comprising a CNN-based or Transformer-based document image classifier that identifies the document type from a scanned image or uploaded file, followed by a named entity extraction stage that populates structured metadata fields such as investigator name, document date, and expiration date. The e-regulatory module employs Computer Vision to verify digital signatures on regulatory records by comparing signature images or cryptographic signature certificates against enrolled investigator profiles stored in the distributed ledger. The e-regulatory module flags regulatory anomalies, such as expired credentials or missing required documents, in real-time and generates alerts on the research site manager computing device. Verified regulatory records are stored on the distributed ledger and made accessible to the sponsor computing device through role-based access controls, enabling remote regulatory oversight without requiring physical document transfer or on-site monitoring visits.

A financial module orchestrated by autonomous AI agents performs real-time financial reconciliation for the clinical research study. The financial module collects verified screening data from the distributed ledger and autonomously cross-references completed research participant visits against digital sponsor contracts to calculate accounts receivable. The digital sponsor contracts may be stored as structured data records that specify billable procedures, per-visit payment amounts, pass-through cost categories, and payment milestones associated with the study protocol. The autonomous AI agents may employ a Reinforcement Learning approach to optimize the reconciliation process, where the state space encodes the set of completed visit procedures, the corresponding contract line items, and any pending discrepancies, the action space comprises matching operations that pair completed procedures with contract line items, and the reward function provides a positive signal for accurate matches confirmed by subsequent audit and a negative signal for mismatches or unresolved discrepancies. Alternatively, the financial module may implement the cross-referencing operation using rule-based matching augmented by a trained classifier that resolves ambiguous mappings between procedure descriptions in the screening data and line item descriptions in the contract. The financial module identifies invoiceable items and pass-through items within the study protocol and maintains a running accounts receivable ledger synchronized with the distributed ledger.

A data review and reports generating module generates screening reports representing the financial value owed to the research site location based on the calculated accounts receivable. The data review and reports generating module provides a Predictive Analytics dashboard accessible on the research site manager computing device and the sponsor computing device. On the research site manager computing device, the dashboard displays screening data summaries and highlights anomalies flagged by an unsupervised machine learning engine. The unsupervised machine learning engine may be implemented as a clustering algorithm such as density-based spatial clustering or an isolation forest algorithm that identifies screening records or visit patterns that deviate from the learned distribution of compliant records. On the sponsor computing device, the dashboard displays real-time trial velocity metrics including enrollment numbers, projected enrollment completion dates, site-selection scorecards based on clinician performance and data integrity metrics, and financial summaries. The dashboard may employ Machine Learning Forecasting models, such as time series regression models or recurrent neural networks trained on historical enrollment data, to generate trial velocity predictions and identify trends in recruitment rates across research sites. An invoice generating module autonomously generates digital invoices based on the screening reports produced by the data review and reports generating module. The invoice generating module formats the invoice data according to sponsor-specified billing templates, transmits the digital invoice to the sponsor computing device over the network, and records the invoice transaction on the distributed ledger to maintain immutable auditability. The invoice generating module may generate and distribute invoices on a periodic schedule synchronized with the verified screening data, or may generate invoices upon the completion of predefined billing milestones specified in the digital sponsor contract.

An NLP-driven feedback module facilitates bidirectional communications between the research site manager computing device and the sponsor computing device. The feedback module receives textual communications such as performance reviews, site queries, and study coordination messages, and performs sentiment analysis and categorization using Natural Language Processing. The sentiment analysis component may be implemented as a fine-tuned Transformer-based text classification model that assigns sentiment polarity scores and topic category labels to each communication. The feedback module aggregates categorized communications to identify performance trends, such as recurring site operational challenges or positive investigator performance patterns, and provides these trends as structured inputs to the autonomous agents within the research process optimizing module. These structured inputs may inform adjustments to the RL agent's scheduling policy, the text reminder generating module's notification strategy, or the data review and reports generating module's anomaly detection thresholds, thereby establishing a closed-loop optimization cycle across the distributed network.

An e-consent and subject retention module manages informed consent and participant engagement throughout the study lifecycle. The e-consent and subject retention module facilitates electronic signing of informed consent documents through the integrated Generative AI chatbot, which presents consent information to participants on the research participant computing device in an interactive conversational format. The AI inference accelerator verifies electronic signatures by validating cryptographic signature certificates or biometric signature samples, and verified consent records are recorded on the distributed ledger. The e-consent and subject retention module further utilizes NLP-based sentiment engines to monitor participant feedback collected through the chatbot and through structured survey instruments. The NLP-based sentiment engines analyze participant responses to detect indicators of disengagement, confusion, or dissatisfaction using a text classification model trained on labeled participant communication datasets. When the sentiment engines detect elevated dropout risk for a participant, the module autonomously deploys targeted engagement communications through the text reminder generating module to address the identified concerns and maintain protocol compliance.

The system may operate in various deployment configurations. In one configuration, the research process optimizing module executes entirely on cloud-hosted infrastructure, with each computing device accessing the module through a web-based or native application interface over the network. In another configuration, portions of the research process optimizing module, such as the primary source data capturing module's OCR pipeline or the integrated Generative AI chatbot's inference model, execute locally on individual computing devices to reduce network latency during real-time data capture and participant interaction, with periodic synchronization of locally generated data to the persistent vector database and distributed ledger through the network. The system may support any number of research participant computing devices, research site manager computing devices, principal investigator computing devices, and sponsor computing devices communicatively coupled through the network, and may manage multiple concurrent clinical research studies with independent protocol criteria, financial contracts, and regulatory document sets stored within the persistent vector database and distributed ledger.

Various implementations of the invention involve the technical field of artificial intelligence systems and methods for clinical research informatics including enabling, via a research participant computing device and a research site manager computing device, the input of research participants' information through an automated enrolment and retention module governed by Deep Learning Neural Networks configured to perform real-time data validation and integrity checks at the point of entry; transmitting the validated research participants' information to a distributed cloud-based AI mesh and a central database over a network for real-time indexing and high-dimensional processing; identifying and ranking qualified research participants using a Transformer-based Large Language Model (LLM) within a list generating module, where the LLM performs semantic vector matching of unstructured medical records, clinician observations, and thoughts against complex study protocol features to generate a list of qualified subjects; facilitating interaction via an integrated Generative AI chatbot on the research participant computing device, enabling qualified participants to input additional data and autonomously schedule study screening appointments; optimizing resource allocation by assigning one or more principal investigators to screen the research participants using a Reinforcement Learning (RL) agent that balances investigator availability, therapeutic expertise, and real-time site resource states; capturing screening data in real-time via a Computer Vision-enhanced primary source data capture module, utilizing Convolutional Neural Networks (CNNs) and Optical Character Recognition (OCR) to autonomously classify laboratory reports and verify investigator sign-offs against ALCOA principles; executing autonomous financial reconciliation by a multi-agent financial module that utilizes Reinforcement Learning to cross-reference captured screening data against digital sponsor contracts, autonomously calculating accounts receivable and eliminating manual billing discrepancies; generating and distributing screening reports through a Generative AI data review module to represent the exact financial value owed to the research site, and automatically sending these reports to an autonomous invoice generating module; autonomously issuing a digital invoice based on the AI-reconciled metrics to a sponsor computing device; and providing a Predictive Analytics dashboard to the sponsor to review the invoice and study health metrics, enabling real-time visibility into enrollment velocity and data integrity and are therefore necessarily rooted in computer technology. For example, the aforementioned steps are inherently computer-based and cannot be performed in the human mind. The present invention amounts to more than merely implementing the generic computer as a tool to gather, analyze, and output data because the steps of the present method, system, or product improve the artificial intelligence systems and methods for clinical research informatics by transforming the way clinical research data is processed and matched by converting unstructured medical narratives, such as clinician observations, progress notes, and procedure notes, into high-dimensional semantic vector embeddings through a Transformer-based encoder and storing them in a persistent vector database with spatial indexing structures that enable sub-linear similarity search. This data transformation is not something a human could perform mentally or through manual organization, because it requires the parallel execution of multi-layer self-attention computations across thousands of token positions to produce dense numerical representations that capture latent semantic relationships between medical concepts. The system then performs semantic vector matching between these participant embeddings and protocol criteria embeddings to identify clinical suitability based on contextual meaning rather than keyword overlap, a computation that relies on the AI inference accelerator executing high-dimensional tensor operations at a scale and speed that has no manual analog.

Beyond this data transformation, the system operates as an integrated, closed-loop autonomous architecture, not a collection of generic tools applied to a business workflow. The Reinforcement Learning agents within the appointment scheduling module and text reminder generating module continuously optimize their policies based on real-time state observations from the distributed network, including site resource utilization, investigator availability, and participant interaction patterns, producing scheduling and engagement decisions that adapt dynamically without human intervention. The primary source data capturing module executes trained Convolutional Neural Networks and an autoencoder-based anomaly detection model in real-time during screening visits to classify laboratory data, verify digital signatures, and flag protocol deviations as they occur, a function that is tightly coupled to the specific hardware configuration of the neural processing hub and AI inference accelerator and that produces a qualitatively different output than manual chart review. The distributed ledger records every verified data capture, sign-off, and financial transaction as cryptographically linked, immutable records, providing an audit integrity mechanism that is structurally impossible to replicate through human recordkeeping. Taken together, these components do not merely automate a series of manual steps on generic hardware; they create an interconnected technical architecture where the output of each module feeds into and conditions the behavior of downstream modules, producing an autonomous orchestration loop that improves its own performance over time and that could not function without the specific processor configurations, trained neural network models, and data structures disclosed in the specification.

Additionally, the steps of the present invention would be impossible to accomplish on pen and paper due to the volume of data being communicated and received over a network in real-time. In particular, the speed at which the steps of the present invention occur to effectuate the disclosed method, system, or product would involve large-scale, continuous wireless communication of such data. That is, the steps of the present method, system, or product are impossible to accomplish on pen and paper, cannot be accomplished as a method of organizing human activity, and amount to significantly more than merely gathering, analyzing, and outputting data.

Implementations of the present invention include implementing (executing, running, or deploying) one or more artificial intelligence models on a computing device wherein the computing device executes the artificial intelligence model's algorithms and mathematical functions on computer hardware using machine learning libraries. The computing device implements the artificial intelligence model when it performs tasks like training, making predictions, applying the model to data, decision-making, classification, or generating outputs based on inputs. In particular, the speed at which an artificial intelligence model analyzes and transforms data to effectuate the disclosed method, system, or product would involve large-scale, continuous transformation of such data. As such, the present invention would be impossible to accomplish on pen and paper or in the human mind due to the volume of data being analyzed and transformed by the artificial intelligence model.

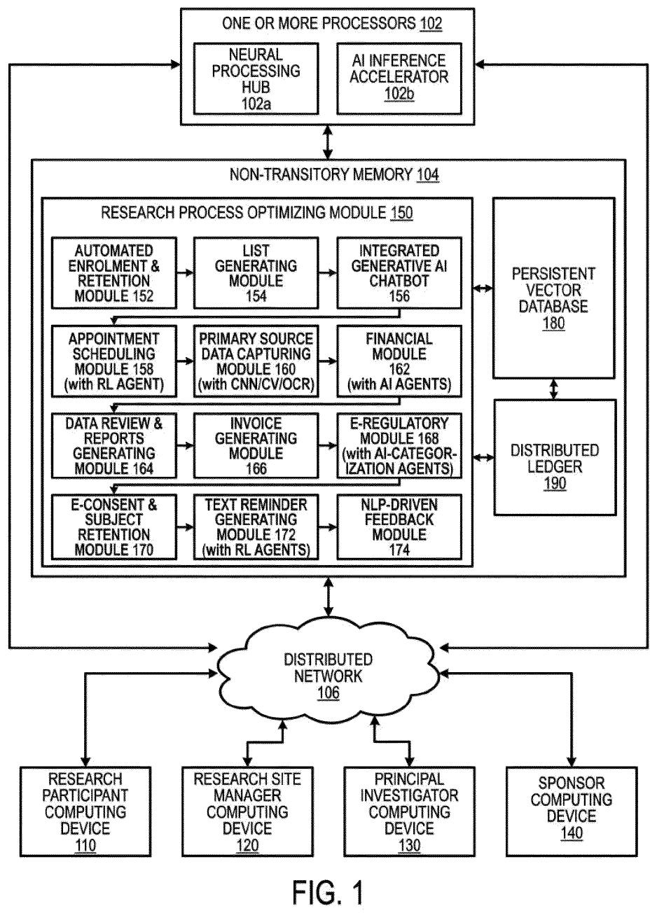

FIG. 1 is a block diagram depicting a system architecture for autonomously optimizing clinical research processes. The system comprises one or more processors 102 including a neural processing hub 102a and an AI inference accelerator 102b, a non-transitory memory 104, a distributed network 106, a research process optimizing module 150, a persistent vector database 180, a distributed ledger 190, and computing devices including a research participant computing device 110, a research site manager computing device 120, a principal investigator computing device 130, and a sponsor computing device 140.

The neural processing hub 102a may be implemented as a multi-core central processing unit or a dedicated neural processing unit with fixed-function hardware for multiply-accumulate operations. The neural processing hub 102a executes orchestration logic for the research process optimizing module 150 and coordinates read and write operations between the non-transitory memory 104, the persistent vector database 180, and the distributed ledger 190. The AI inference accelerator 102b may be implemented as a graphics processing unit, a field-programmable gate array, or an application-specific integrated circuit designed to accelerate forward-pass computations of trained neural network models. The AI inference accelerator 102b handles tensor operations required by the Transformer-based Large Language Model within the list generating module 154, the Convolutional Neural Networks within the primary source data capturing module 160, the Reinforcement Learning agents within the appointment scheduling module 158 and the text reminder generating module 172, and biometric verification and electronic signature validation operations performed across the system.

The non-transitory memory 104 may comprise volatile memory such as random access memory providing a low-latency runtime environment, and non-volatile memory such as solid-state drives providing persistent storage for encrypted medical data, trained model weights, and regulatory records. The non-transitory memory 104 hosts the executable instructions for each sub-module and serves as working memory for intermediate computation results.

The research process optimizing module 150 resides within the non-transitory memory 104 and comprises twelve sub-modules: an automated enrolment and retention module 152, a list generating module 154, an integrated Generative AI chatbot 156, an appointment scheduling module 158 with a Reinforcement Learning agent, a primary source data capturing module 160 with CNN, Computer Vision, and OCR capabilities, a financial module 162 with autonomous AI agents, a data review and reports generating module 164, an invoice generating module 166, an e-regulatory module 168 with AI-categorization agents, an e-consent and subject retention module 170, a text reminder generating module 172 with Reinforcement Learning agents, and an NLP-driven feedback module 174.

The automated enrolment and retention module 152 receives research participants'information, which may comprise unstructured health history, clinical medical reports, longitudinal diagnosis reports, medication lists, progress notes, consultation reports, procedure notes, laboratory reports, and medical imaging data. The module 152 parses this information using a Natural Language Processing model comprising tokenization, named entity recognition, relation extraction, and negation detection. The module 152 generates high-dimensional semantic vector embeddings using a Transformer-based encoder model and stores the embeddings in the persistent vector database 180.

The list generating module 154 executes a Transformer-based Large Language Model to generate a ranked list of qualified research participants. The module 154 transforms protocol inclusion and exclusion criteria into semantic vector embeddings and performs semantic vector matching by computing similarity scores, which may use cosine similarity or Euclidean distance, between the protocol criteria embeddings and participant profile embeddings stored in the persistent vector database 180. The persistent vector database 180 may employ approximate nearest neighbor search algorithms such as hierarchical navigable small world graphs for sub-linear query complexity. The module 154 ranks participants by aggregate similarity scores and applies threshold filtering.

The integrated Generative AI chatbot 156 may be implemented as an autoregressive Transformer-based language model conditioned on a conversational context window comprising prior messages, protocol-specific reference material, and system instructions. The chatbot 156 provides protocol-specific clinical decision support, assists participants through electronic informed consent processes, collects additional health context, and facilitates scheduling by transmitting requests to the appointment scheduling module 158.

The appointment scheduling module 158 employs a Reinforcement Learning agent, which may be implemented as a deep Q-network or policy gradient agent, operating over a state space encoding pending appointments, investigator availability, therapeutic specializations, participant complexity, and facility utilization. The action space comprises investigator-to-appointment assignments, and the reward function provides positive signals for completed screenings within target time windows and negative signals for conflicts or cancellations.

The primary source data capturing module 160 captures screening data in real-time on the principal investigator computing device 130. The module 160 executes CNNs and Computer Vision for automated OCR comprising a text detection stage using a CNN-based object detection model and a text recognition stage using a CNN encoder coupled with a recurrent neural network decoder or Transformer-based sequence model. The module 160 applies domain-specific post-processing to extract structured data fields and may employ a CNN-based image classification model for medical imaging. The module 160 employs deep learning anomaly detection, which may be implemented as an autoencoder neural network, to flag protocol deviations when reconstruction error exceeds a configurable threshold. Verified screening data and digital sign-offs are recorded on the distributed ledger 190 to ensure adherence to ALCOA principles.

The financial module 162 collects verified screening data from the distributed ledger 190 and cross-references completed visits against digital sponsor contracts specifying billable procedures, payment amounts, pass-through costs, and milestones. The autonomous AI agents may employ Reinforcement Learning or rule-based matching augmented by a trained classifier. The module 162 calculates accounts receivable and transmits financial data to the data review and reports generating module 164.

The data review and reports generating module 164 generates screening reports representing the financial value owed to the research site. The module 164 provides a Predictive Analytics dashboard on the research site manager computing device 120, displaying anomalies flagged by an unsupervised machine learning engine such as density-based spatial clustering or an isolation forest algorithm, and on the sponsor computing device 140, displaying trial velocity metrics, site-selection scorecards, and financial summaries. The module 164 may employ Machine Learning Forecasting models to generate trial velocity predictions.

The invoice generating module 166 autonomously generates digital invoices based on screening reports, formats them according to sponsor-specified billing templates, transmits them to the sponsor computing device 140, and records transactions on the distributed ledger 190. Invoices may be generated periodically or upon completion of predefined billing milestones.

The e-regulatory module 168 utilizes Autonomous AI-Categorization Agents comprising a document classification pipeline and named entity extraction to ingest, digitize, classify, and store regulatory records. The module 168 employs Computer Vision to verify digital signatures and flags regulatory anomalies in real-time. Verified records are stored on the distributed ledger 190 with role-based access controls.

The e-consent and subject retention module 170 facilitates electronic informed consent through the chatbot 156 and utilizes NLP-based sentiment engines to detect indicators of disengagement or dropout risk. When elevated risk is detected, the module 170 triggers the text reminder generating module 172 to deploy targeted engagement communications.

The text reminder generating module 172 utilizes Predictive Sentiment Analysis, which may be implemented as a fine-tuned Transformer-based classification model, and RL agents that optimize a notification policy over participant interaction history, communication timing, and enrollment stage. The module 172 dynamically adjusts reminder cadence based on ongoing interaction patterns.

The NLP-driven feedback module 174 facilitates bidirectional communications between the research site manager computing device 120 and the sponsor computing device 140, performing sentiment analysis and categorization to identify performance trends. These trends inform adjustments to scheduling policies, notification strategies, and anomaly detection thresholds, establishing a closed-loop optimization cycle.

The persistent vector database 180 stores and indexes participant information as high-dimensional semantic vector embeddings and may employ approximate nearest neighbor search algorithms. The persistent vector database 180 may also store protocol criteria embeddings, investigator profile embeddings, and site capability embeddings for cross-trial suitability analysis.

The distributed ledger 190 maintains an append-only, cryptographically linked chain of records storing verified clinical data, sign-offs, regulatory documents, consent records, financial transactions, and invoices. Each record includes a cryptographic hash of the preceding record, a timestamp, and the recorded data. The distributed ledger 190 provides role-based access controls and ensures adherence to ALCOA principles.

The distributed network 106 may comprise Ethernet, Wi-Fi, cellular networks including 5G, or combinations thereof. The computing devices 110, 120, 130, and 140 may be smartphones, tablets, laptops, or desktop computers. The research participant computing device 110 serves as the interface for chatbot interaction, health record submission, notification receipt, consent, and scheduling. The research site manager computing device 120 serves as the interface for participant data input, regulatory document ingestion, analytics review, and feedback. The principal investigator computing device 130 serves as the interface for screening data capture, compliance guidance, and digital sign-offs. The sponsor computing device 140 serves as the interface for invoice review, trial velocity monitoring, regulatory document access, and bidirectional feedback.

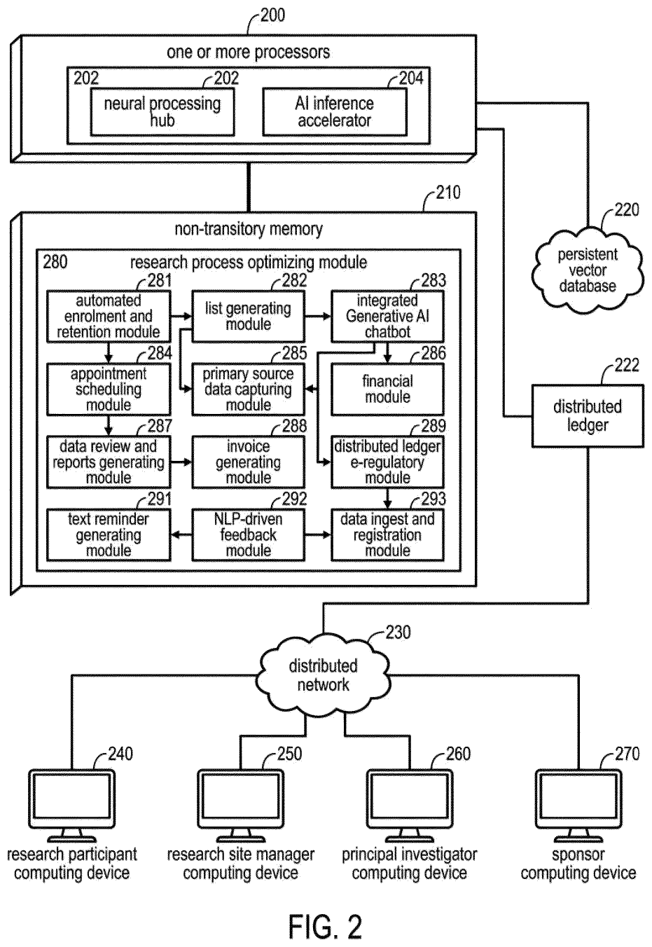

FIG. 2 is a block diagram depicting an alternative embodiment comprising one or more processors 200 including a neural processing hub 202 and an AI inference accelerator 204, a non-transitory memory 210, a research process optimizing module 280, a persistent vector database 220, a distributed ledger 222, a distributed network 230, and computing devices 240, 250, 260, and 270. FIG. 2 differs from FIG. 1 in that the persistent vector database 220 and distributed ledger 222 are depicted as external data stores outside the non-transitory memory 210 boundary, and in that the research process optimizing module 280 includes a data ingest and registration module 292 as a distinct sub-module.

The research process optimizing module 280 comprises thirteen sub-modules: an automated enrolment and retention module 281, a list generating module 282, an integrated Generative AI chatbot 283, an appointment scheduling module 284, a primary source data capturing module 285, a financial module 286, a data review and reports generating module 287, an invoice generating module 288, a distributed ledger e-regulatory module 289, a text reminder generating module 290, an NLP-driven feedback module 291, a data ingest and registration module 292, and an e-consent and subject retention module distributed across the chatbot 283, the text reminder generating module 290, and the NLP-driven feedback module 291. Each sub-module operates in the same manner as the corresponding module described in connection with FIG. 1, with the same NLP pipelines, Transformer-based encoders, RL agents, CNN-based OCR pipelines, anomaly detection autoencoders, financial reconciliation agents, and sentiment analysis models.

The data ingest and registration module 292 authenticates users through biometric AI and identity verification agents using trained CNN classifiers for fingerprint, facial, or iris recognition. Upon authentication, the module 292 stores identity credentials on the distributed ledger 222 and assigns role-based access permissions. The module 292 maps authenticated profiles into the persistent vector database 220 as semantic profile embeddings for role-based orchestration.

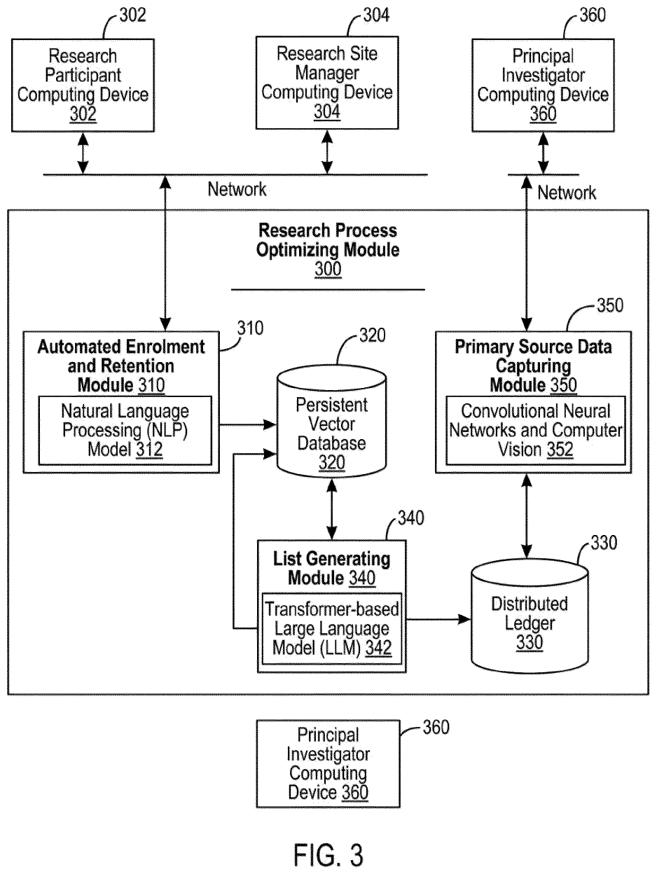

FIG. 3 is a block diagram depicting a detailed view of the data ingestion, transformation, and semantic matching pipeline within the research process optimizing module 300. The diagram illustrates data flow from the research participant computing device 302 and the research site manager computing device 304 through the automated enrolment and retention module 310, the persistent vector database 320, and the list generating module 340 to the principal investigator computing device 360, alongside a parallel pathway from the principal investigator computing device 360 through the primary source data capturing module 350 to the distributed ledger 330.

The automated enrolment and retention module 310 contains an NLP Model 312 that parses unstructured medical records through tokenization, named entity recognition, relation extraction, and negation detection, and generates high-dimensional semantic vector embeddings using a Transformer-based encoder. The embeddings are stored in the persistent vector database 320, which indexes them using approximate nearest neighbor search algorithms for efficient similarity retrieval.

The list generating module 340 contains a Transformer-based Large Language Model (LLM) 342 that transforms protocol criteria into semantic vector embeddings and performs semantic vector matching against participant embeddings in the persistent vector database 320. The module 340 outputs a ranked list of qualified participants to the principal investigator computing device 360.

The primary source data capturing module 350 contains Convolutional Neural Networks and Computer Vision 352 for automated OCR and deep learning anomaly detection. The module 350 captures and verifies screening data from the principal investigator computing device 360 and records verified data on the distributed ledger 330. FIG. 3 isolates the two primary data transformation operations: the semantic embedding and matching pipeline and the computer vision-based capture and verification pipeline.

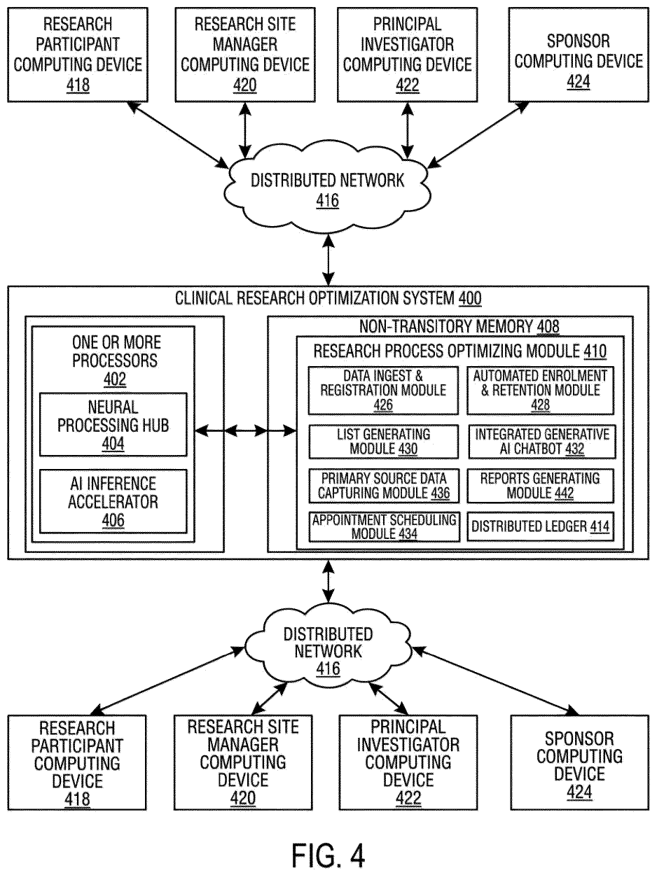

FIG. 4 is a block diagram depicting a clinical research optimization system 400 illustrating bidirectional data flow. Computing devices 418, 420, 422, and 424 appear at both the top and bottom of the diagram, connected through two instances of the distributed network 416, illustrating a continuous processing loop in which input data is transformed within the system and returned as actionable outputs.

The clinical research optimization system 400 encloses one or more processors 402 comprising a neural processing hub 404 and an AI inference accelerator 406, and a non-transitory memory 408 storing the research process optimizing module 410. The module 410 comprises a data ingest and registration module 426, an automated enrolment and retention module 428, a list generating module 438, an integrated Generative AI chatbot 432, an appointment scheduling module 434, a primary source data capturing module 436, a reports generating module 442, and a distributed ledger 414 positioned within the module boundary. Each sub-module operates in the same manner as the corresponding module described in connection with the preceding figures.

FIG. 4 differs from the preceding figures in that it introduces the system 400 as a unified system-level boundary, depicts the distributed ledger 414 within the module boundary illustrating local deployment, and depicts computing devices at both top and bottom to emphasize the closed-loop architecture.

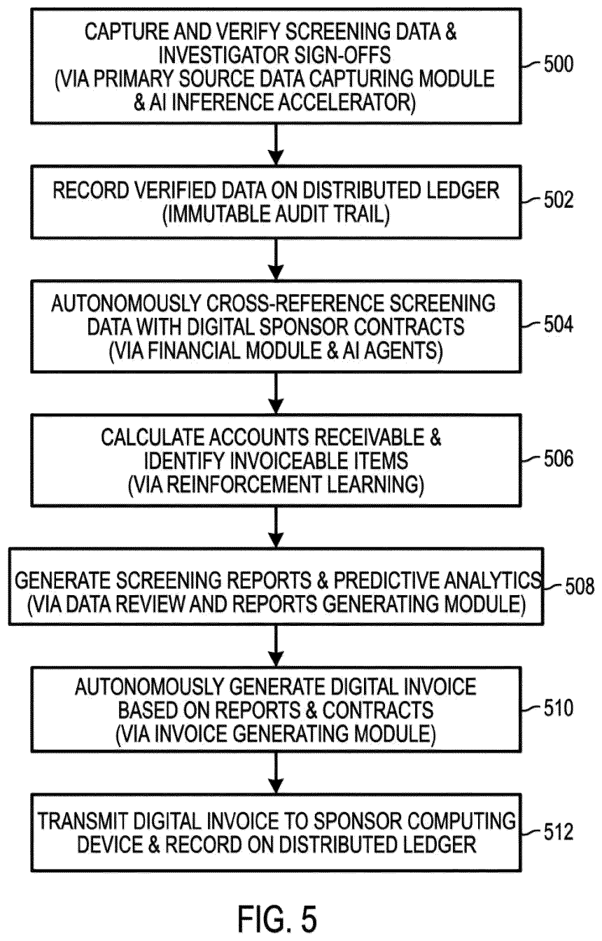

FIG. 5 is a flow diagram depicting a method for autonomous financial reconciliation and invoice generation comprising steps 500 through 512.

At step 500, the primary source data capturing module captures and verifies screening data and investigator sign-offs using CNNs, Computer Vision, automated OCR, and deep learning anomaly detection. The AI inference accelerator verifies digital sign-offs by validating cryptographic signature certificates or biometric samples.

At step 502, the system records verified data on the distributed ledger as an immutable audit trail, ensuring the clinical data underlying subsequent financial calculations is ALCOA-compliant.

At step 504, the financial module and autonomous AI agents cross-reference verified screening data against digital sponsor contracts specifying billable procedures, payment amounts, pass-through costs, and milestones. The agents may employ Reinforcement Learning or rule-based matching augmented by a trained classifier.

At step 506, the financial module calculates accounts receivable and identifies invoiceable items including per-visit payments, milestone payments, and pass-through costs. RL agents refine matching accuracy over successive billing cycles based on sponsor approval or rejection feedback.

At step 508, the data review and reports generating module generates screening reports and predictive analytics using Machine Learning Forecasting models, and provides a Predictive Analytics dashboard displaying anomalies, trial velocity metrics, and financial summaries.

At step 510, the invoice generating module autonomously generates a digital invoice formatted according to sponsor-specified billing templates. At step 512, the module transmits the invoice to the sponsor computing device and records the transaction on the distributed ledger, establishing an immutable financial trail traceable to the verified screening data from step 502.

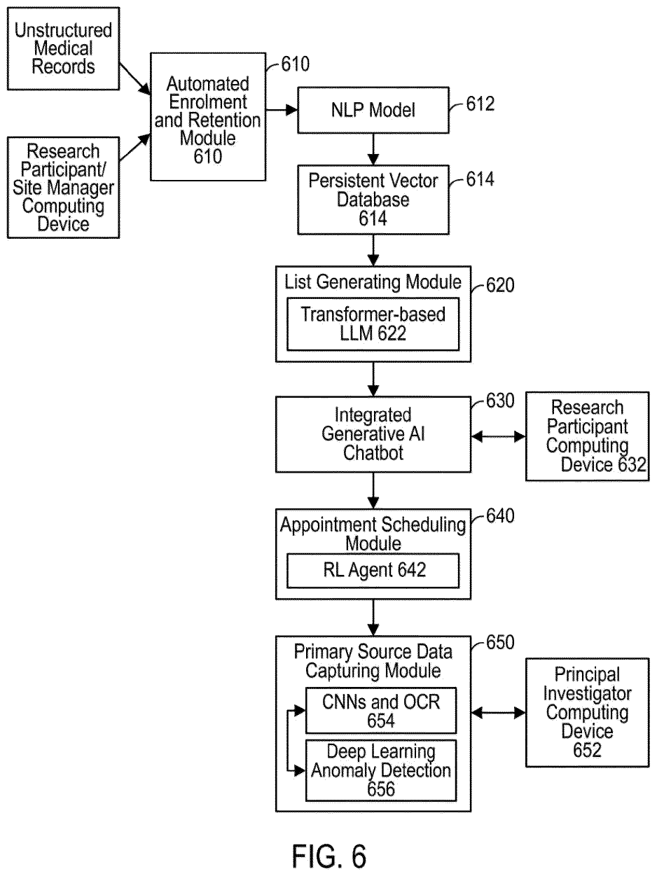

FIG. 6 is a flow diagram depicting the end-to-end clinical data processing pipeline as a vertical sequence of processing stages. External inputs of unstructured medical records and participant/site manager computing devices flow into the automated enrolment and retention module 610, which contains an NLP Model 612 that parses unstructured records and generates high-dimensional semantic vector embeddings stored in the persistent vector database 614.

The list generating module 620, containing a Transformer-based LLM 622, performs semantic vector matching against protocol criteria to generate a ranked list of qualified participants. The integrated Generative AI chatbot 630 interacts with participants through the research participant computing device 632 to provide clinical decision support, facilitate consent, and collect scheduling requests.

The appointment scheduling module 640, containing an RL Agent 642, autonomously assigns principal investigators based on therapeutic expertise and real-time site resource states. The primary source data capturing module 650 contains two sub-components: CNNs and OCR 654 for automated document classification and verification, and Deep Learning Anomaly Detection 656 implemented as an autoencoder that flags protocol deviations when reconstruction error exceeds a configurable threshold. The module 650 captures screening data from the principal investigator computing device 652 and records verified data on the distributed ledger. FIG. 6 isolates the serial dependency chain from unstructured data ingestion to verified screening records.

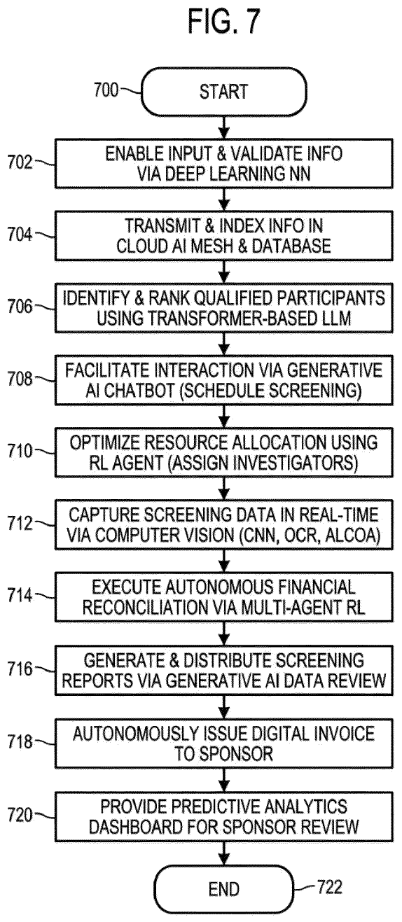

FIG. 7 is a flow diagram depicting an end-to-end method 700 from a start step 700 through steps 702 to 720 and concluding at an end step 722.

At step 702, the system validates incoming participant information via Deep Learning Neural Networks. At step 704, the system parses validated information using NLP, generates semantic vector embeddings, and indexes them in a persistent vector database on a cloud server. At step 706, the list generating module performs semantic vector matching using a Transformer-based LLM to rank qualified participants. At step 708, the Generative AI chatbot facilitates participant engagement and scheduling. At step 710, an RL agent assigns principal investigators based on real-time site conditions. At step 712, the primary source data capturing module captures and verifies screening data using CNNs, OCR, and anomaly detection, recording verified data on the distributed ledger. At step 714, the financial module performs autonomous reconciliation against digital sponsor contracts. At step 716, screening reports and predictive analytics are generated. At step 718, a digital invoice is autonomously generated and transmitted to the sponsor. At step 720, a Predictive Analytics dashboard enables sponsor review of the invoice and study health metrics. The method may repeat from step 702 to establish a continuous processing loop.

FIG. 7 combines the clinical pipeline of FIG. 6 and the financial pipeline of FIG. 5 into a unified end-to-end method, and additionally includes data validation at entry, cloud indexing, and a sponsor dashboard review step.

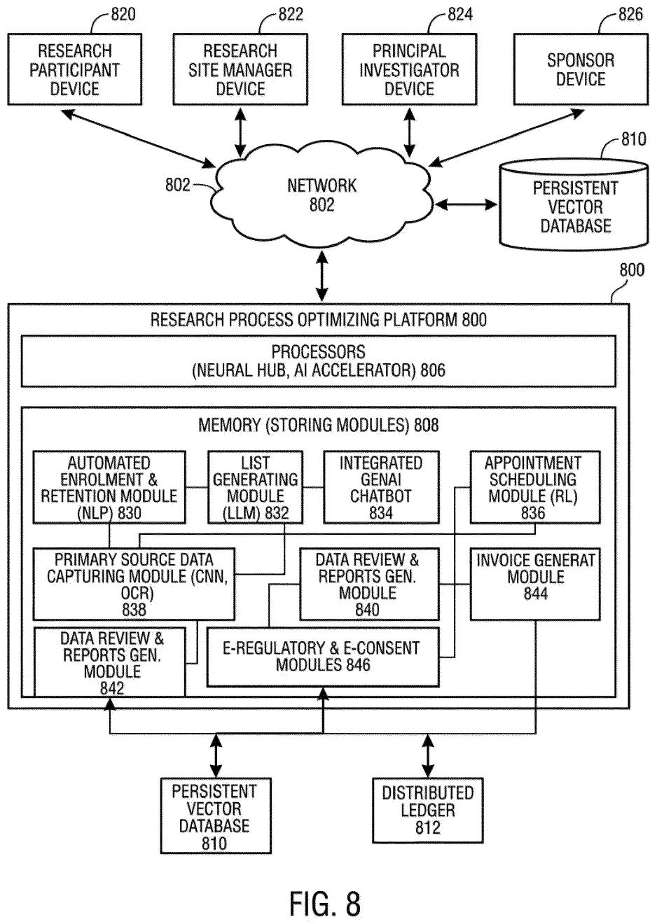

FIG. 8 is a block diagram depicting a research process optimizing platform 800 illustrating a hybrid deployment architecture. The platform 800 encloses processors 806 comprising a neural hub and AI accelerator, memory 808 storing sub-modules, a persistent vector database 810, and a distributed ledger 812. A second instance of the persistent vector database 810 is depicted externally, coupled to network 802, illustrating hybrid cloud and local storage with synchronization.

The memory 808 encloses an automated enrolment and retention module 830 (NLP), a list generating module 832 (LLM), an integrated GenAI chatbot 834, an appointment scheduling module 836 (RL), a primary source data capturing module 838 (CNN, OCR), two data review and reports generating modules 840 and 842, an invoice generating module 844, and combined e-regulatory and e-consent modules 846. Computing devices 820, 822, 824, and 826 connect through network 802.

The two data review and reports generating modules 840 and 842 illustrate role-specific dashboard configurations, with module 840 providing sponsor-facing financial and trial velocity reporting and module 842 providing site manager-facing operational and data quality reporting. The combined e-regulatory and e-consent modules 846 illustrate an alternative configuration in which regulatory compliance and participant retention operations share common document processing, signature verification, and distributed ledger infrastructure.

FIG. 8 differs from the preceding figures in introducing a platform-level boundary, depicting hybrid vector database deployment, providing role-specific reporting modules, and combining e-regulatory and e-consent functions, demonstrating deployment flexibility while maintaining autonomous orchestration capabilities.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A system for autonomously optimizing clinical research participant identification and recruitment, comprising:
one or more processors comprising a neural processing hub and an AI inference accelerator configured for high-dimensional tensor operations;
a non-transitory memory coupled to the one or more processors, the memory storing a research process optimizing module configured to be executed across a distributed network of computing devices including a research participant computing device, a research site manager computing device, a principal investigator computing device, and a sponsor computing device;
a persistent vector database hosted on a cloud server and communicatively coupled to the distributed network over a network, the persistent vector database configured to store and index research participants' information as high-dimensional semantic vector embeddings;
wherein the research process optimizing module comprises:
an automated enrolment and retention module configured to: (a) receive, from at least one of the research participant computing device and the research site manager computing device, one or more research participants' information comprising unstructured health history and medical records; and (b) parse the unstructured health history and medical records using a Natural Language Processing (NLP) model to transform the one or more research participants' information into high-dimensional semantic vector embeddings and store the high-dimensional semantic vector embeddings in the persistent vector database; and
a list generating module configured to execute a Transformer-based Large Language Model (LLM) on the one or more processors to: (a) perform semantic feature extraction on the high-dimensional semantic vector embeddings stored in the persistent vector database, wherein the semantic feature extraction comprises analyzing clinician documentation including observations, thoughts, and actions from progress notes, consultation reports, and procedure notes; and (b) generate a ranked list of qualified research participants by performing semantic vector matching of the extracted semantic features against one or more protocol features comprising inclusion criteria and exclusion criteria of a clinical research study, thereby identifying clinical suitability beyond keyword-based matching.

2. The system of claim 1, wherein the automated enrolment and retention module is further configured to transmit the high-dimensional semantic vector embeddings to the persistent vector database via autonomous AI agents operating within a distributed cloud-based AI mesh comprising the cloud server and a central database, wherein the transmission is encrypted and validated for protocol-specific data integrity in real-time.

3. The system of claim 2, wherein the persistent vector database is further configured to enable Predictive Analytics models executed by the AI inference accelerator to forecast recruitment velocity based on historical enrollment patterns and to identify research participants at high risk of dropout before completion of the clinical research study.

4. The system of claim 1, wherein the one or more research participants' information further comprises longitudinal diagnosis reports, medication lists, and medical imaging data, and wherein the NLP model is configured to extract semantic features from the longitudinal diagnosis reports and medication lists in conjunction with the clinician documentation to determine protocol suitability across multiple concurrent clinical research studies.

5. The system of claim 1, further comprising a data ingest and registration module configured to authenticate the research participant, the principal investigator, and the sponsor using Biometric AI and identity verification agents, the data ingest and registration module storing authenticated identity credentials as immutable records within a distributed ledger to ensure auditable access control across the distributed network.

6. A system for providing an autonomously optimized multi-agent AI process in clinical research, comprising:
one or more processors comprising a neural processing hub configured for parallel execution of neural network inference and a communication path coupling the one or more processors to a non-transitory memory and a network interface;
the non-transitory memory storing a research process optimizing module, the research process optimizing module comprising:
an automated enrolment and retention module configured to receive one or more research participants' information comprising unstructured medical records from at least one of a research participant computing device and a research site manager computing device over a network, and to parse the unstructured medical records using a Natural Language Processing (NLP) model to transform the unstructured medical records into high-dimensional semantic vector embeddings stored in a persistent vector database;
a list generating module configured to execute a Transformer-based Large Language Model (LLM) to perform semantic vector matching of the high-dimensional semantic vector embeddings against inclusion criteria and exclusion criteria of a clinical research study protocol to generate a ranked list of qualified research participants;
an integrated Generative AI chatbot configured to interact with the qualified research participants via the research participant computing device to provide protocol-specific clinical decision support and to facilitate electronic signing of informed consent documents;
an appointment scheduling module configured to employ a Reinforcement Learning (RL) agent to autonomously assign one or more principal investigators for screening the qualified research participants, wherein the RL agent balances investigator therapeutic expertise, investigator availability, and real-time site resource states to optimize resource allocation; and
a primary source data capturing module configured to capture screening data of the one or more research participants on the principal investigator computing device in real-time, wherein the primary source data capturing module executes Convolutional Neural Networks (CNNs) and Computer Vision for automated Optical Character Recognition (OCR) to classify and verify laboratory reports, medical imaging, and investigator digital sign-offs, and wherein the primary source data capturing module employs deep learning anomaly detection to flag protocol deviations in real-time to ensure adherence to ALCOA principles.

7. The system of claim 6, wherein the integrated Generative AI chatbot is further configured to operate as a virtual assistant that answers protocol-specific queries from the qualified research participants and assists the qualified research participants through a process of reading and providing electronic signatures on informed consent documents, wherein the electronic signatures are verified by the AI inference accelerator and recorded on a distributed ledger.

8. The system of claim 6, further comprising a text reminder generating module driven by Reinforcement Learning (RL) agents, wherein the RL agents are configured to autonomously determine an optimal timing and a semantic tone of notifications sent to the research participant computing device based on participant interaction history, and wherein the text reminder generating module utilizes Predictive Sentiment Analysis to tailor the notifications to maximize participant retention and enrollment velocity.

9. The system of claim 6, wherein the appointment scheduling module is further configured to enable the qualified research participants to opt-in to AI-managed automated reminders, wherein the RL agent dynamically adjusts a scheduling frequency and a reminder cadence based on real-time site resource states and participant interaction history.

10. The system of claim 6, wherein the primary source data capturing module is further configured to provide the principal investigator with real-time protocol compliance guidance during history and physical examinations by executing multi-modal AI inference on the one or more processors to validate the screening data against protocol-specific requirements as the screening data is captured.

11. The system of claim 6, further comprising a distributed ledger e-regulatory module configured to utilize Autonomous AI-Categorization Agents to ingest, digitize, classify, and store regulatory records including financial disclosure forms, delegation logs, and curriculum vitae within an electronic storage system, wherein the Autonomous AI-Categorization Agents employ Computer Vision to verify digital signatures on the regulatory records and flag regulatory anomalies in real-time, and wherein verified regulatory records are stored on a distributed ledger accessible to the sponsor computing device.

12. A system for providing an autonomously optimized multi-agent AI process spanning participant recruitment through financial reconciliation in clinical research, comprising:
one or more processors comprising a neural processing hub and an AI inference accelerator, coupled via a communication path to a non-transitory memory, a persistent vector database, a distributed ledger, and a network interface connecting a distributed network of computing devices including a research participant computing device, a research site manager computing device, a principal investigator computing device, and a sponsor computing device;
the non-transitory memory storing a research process optimizing module comprising:
an automated enrolment and retention module configured to receive one or more research participants' information comprising unstructured health history and medical records from at least one of the research participant computing device and the research site manager computing device, and to parse the unstructured health history and medical records using a Natural Language Processing (NLP) model to generate high-dimensional semantic vector embeddings stored in the persistent vector database;
a list generating module configured to execute a Transformer-based Large Language Model (LLM) on the one or more processors to perform semantic vector matching of the high-dimensional semantic vector embeddings against inclusion criteria and exclusion criteria of a clinical research study protocol to generate a ranked list of qualified research participants;
an appointment scheduling module comprising an integrated Generative AI chatbot and a Reinforcement Learning (RL) agent, the integrated Generative AI chatbot configured to interact with the qualified research participants via the research participant computing device to facilitate scheduling of screening appointments, the RL agent configured to autonomously assign one or more principal investigators based on investigator therapeutic expertise and real-time site resource states;
a primary source data capturing module configured to capture screening data in real-time on the principal investigator computing device, the primary source data capturing module utilizing Convolutional Neural Networks (CNNs) and Computer Vision for automated Optical Character Recognition (OCR) to classify and verify laboratory reports and medical imaging, and employing deep learning anomaly detection to flag protocol deviations, wherein verified screening data and investigator digital sign-offs are recorded on the distributed ledger to ensure adherence to ALCOA principles;
a financial module orchestrated by autonomous AI agents, the financial module configured to: (a) collect the verified screening data in real-time from the distributed ledger; (b) autonomously cross-reference completed research participant visits recorded on the distributed ledger against digital sponsor contracts using Reinforcement Learning to calculate accounts receivable and eliminate manual billing errors;
a data review and reports generating module configured to: (a) generate one or more screening reports representing a financial value owed to a research site location based on the calculated accounts receivable; and (b) provide a Predictive Analytics dashboard on the research site manager computing device for the site manager to review screening data and identify anomalies flagged by an unsupervised machine learning engine, and on the sponsor computing device for the sponsor to review the one or more screening reports and real-time trial velocity metrics; and
an invoice generating module configured to autonomously generate a digital invoice based on the one or more screening reports and transmit the digital invoice to the sponsor computing device over the network, wherein the digital invoice is recorded on the distributed ledger to ensure immutable auditability.

13. The system of claim 12, wherein the financial module is further configured to identify invoiceable items and pass-through items within the clinical research study protocol using the Autonomous AI-Categorization Agents, and wherein the invoice generating module autonomously generates and distributes digital invoices on a periodic schedule synchronized with the verified screening data recorded on the distributed ledger.

14. The system of claim 12, further comprising a real-time metrics updating module configured to synchronize principal investigator performance metrics and research site metrics across the distributed network via the neural processing hub, the real-time metrics updating module utilizing Machine Learning Forecasting to provide the sponsor computing device with real-time enrollment numbers, trial velocity predictions, and site-selection scorecards based on clinician performance and data integrity metrics derived from the distributed ledger.

15. The system of claim 12, further comprising an e-consent and subject retention module configured to: (a) facilitate electronic signing of informed consent documents via the integrated Generative AI chatbot, wherein electronic signatures are verified by the AI inference accelerator and recorded on the distributed ledger; and (b) utilize NLP-based sentiment engines to monitor participant feedback and autonomously deploy engagement communications to maintain protocol compliance and reduce participant dropout rates.

16. The system of claim 12, further comprising an NLP-driven feedback module configured to: (a) receive bidirectional communications between the research site manager computing device and the sponsor computing device; (b) perform sentiment analysis and categorization on the bidirectional communications using Natural Language Processing to identify performance trends; and (c) provide actionable insights to autonomous agents within the research process optimizing module to optimize trial orchestration and site-sponsor communication.

17. A method for providing an autonomously optimized multi-agent process in clinical
research, comprising:
enabling, via a research participant computing device and a research site manager computing device, the input of research participants' information through an automated enrolment and retention module governed by Deep Learning Neural Networks configured to perform real-time data validation and integrity checks at the point of entry;
transmitting the validated research participants' information to a distributed cloud-based AI mesh and a central database over a network for real-time indexing and high-dimensional processing
identifying and ranking qualified research participants using a Transformer-based Large Language Model (LLM) within a list generating module, where the LLM performs semantic vector matching of unstructured medical records, clinician observations, and thoughts against complex study protocol features to generate a list of qualified subjects;
facilitating interaction via an integrated Generative AI chatbot on the research participant computing device, enabling qualified participants to input additional data and autonomously schedule study screening appointments;
optimizing resource allocation by assigning one or more principal investigators to screen the research participants using a Reinforcement Learning (RL) agent that balances investigator availability, therapeutic expertise, and real-time site resource states;
capturing screening data in real-time via a Computer Vision-enhanced primary source data capture module, utilizing Convolutional Neural Networks (CNNs) and Optical Character Recognition (OCR) to autonomously classify laboratory reports and verify investigator sign-offs against ALCOA principles;
executing autonomous financial reconciliation by a multi-agent financial module that utilizes Reinforcement Learning to cross-reference captured screening data against digital sponsor contracts, autonomously calculating accounts receivable and eliminating manual billing discrepancies;
generating and distributing screening reports through a Generative AI data review module to represent the exact financial value owed to the research site, and automatically sending these reports to an autonomous invoice generating module;
autonomously issuing a digital invoice based on the AI-reconciled metrics to a sponsor computing device; and
providing a Predictive Analytics dashboard to the sponsor to review the invoice and study health metrics, enabling real-time visibility into enrollment velocity and data integrity.

18. The method of claim 17, comprising a step of generating the list of qualified research participants using a Transformer-based Large Language Model (LLM) configured to perform semantic vector matching; wherein the LLM autonomously extracts and indexes semantic features from unstructured medical narratives, including clinician documentation of observations, thoughts, and actions, to identify clinical suitability beyond keyword-based inclusion and exclusion criteria.

19. A computer program product comprising a non-transitory computer-readable medium having a computer-readable program code embodied therein to be executed by one or more processors, said program code including instructions to:
enable, via a Multi-Agent AI Orchestration layer, a research participant and a site manager to input information through an enrolment and retention module, wherein said information is autonomously validated for protocol-specific data integrity in real-time;
transform the research participants' information into high-dimensional vector embeddings and transmit said embeddings to a cloud-based vector database for semantic indexing and cross-trial suitability analysis;
generate a list of qualified research participants by executing a neural network inference engine that parses the central database to match participant profiles against protocol features through natural language understanding (NLU);
facilitate participant engagement through an integrated Generative AI chatbot on the research participant computing device, allowing participants to provide additional health context and autonomously schedule screening appointments via a Reinforcement Learning (RL) scheduler;
optimize site operations by assigning one or more principal investigators to screen the research participants using an Autonomous Resource Agent that evaluates real-time site capacity and therapeutic expertise;
capture clinical screening data in real-time via a Computer Vision-enhanced capture module, utilizing Convolutional Neural Networks (CNNs) to autonomously classify medical records and verify investigator sign-offs against ALCOA principles;
execute autonomous financial reconciliation by a Multi-Modal Financial Agent configured to cross-reference completed study visit procedures with digital sponsor contracts, thereby autonomously calculating accounts receivable and generating financial reports without human data entry;
autonomously generate and transmit a digital invoice to a sponsor computing device based on the AI-reconciled study metrics, enabling the sponsor to review the invoice via a Predictive Analytics dashboard that displays real-time trial velocity and audit-readiness metrics.
